(19) United States
(12) Patent Application Publication
(10) Pub. No.: US 2003/0099672 A1
(43) Pub. Date: May 29, 2003

(54) Pneumococcus polysaccharide conjugates for use as vaccine against tetanus an diphtheria

(76) Inventor: Dominique Schultz, Lyon (FR)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/221,978

(22) PCT Filed: Mar. 19, 2001
(86) PCT No.: PCT/FR01/00820

(30) Foreign Application Priority Data
Mar. 17, 2000 (FR) 00/03407

(51) Int. Cl. A61K 39/05; A61K 39/08; A61K 39/09; C08B 37/00

(52) U.S. Cl. 424/239.1; 424/238.1; 424/244.1; 536/53

(57) ABSTRACT

The invention relates to the use of a composition comprising n Streptococcus pneumoniae polysaccharides conjugated to the tetanus toxoid and p Streptococcus pneumoniae polysaccharides conjugated to the diphtheria toxoid, for manufacturing a vaccine which protects against Clostridium tetani and/or Corynebacterium diphtheriae infections in which:

(1) n and p are other than 1, with p being, however, ≦15,

(2) 2≦n+p≦38,

(3) the total amount of conjugated toxoid present in one vaccine dose is sufficient to induce protection against Clostridium tetani and/or Corynebacterium diphtheriae infections.

The present invention relates to the use of vaccine combinations for preventing tetanus and/or diphtheria.

### STATE OF THE ART

In multivalent vaccine compositions, although there are many advantages in mutually combining the antigens so as to confer protection against several pathogens, negative interactions between the antigens may exist, the consequence of which is a relative drop in the immunogenicity of one or more components. This risk is all the greater given that the number of antigens, also called “valences”, is considerable.

Multivalent vaccines are known which comprise in particular diphtheria and tetanus valencies. Combining diphtheria, tetanus and whooping cough antigens with those of the polio virus leads to a decrease in the immune response to whooping cough.

Vaccine combinations are also known in which a polysaccharide antigen is coupled to a carrier protein such as the tetanus toxoid or the diphtheria toxoid. Some authors have wondered about the immune response obtained with respect to the carrier protein. Homayoun S. et al., APMIS (1998); 106; 526-534 has observed that the specific antibody response against tetanus toxoid varies considerably depending on whether this protein is coupled to a low molecular weight or high molecular weight capsular polysaccharide of hemophilus influenzae type b. Anderson P. et al., J. Immunol. (1989), 142, 2464-2468 has also shown that the size of the polysaccharide may also have an influence on the response to the diphtheria toxoid when this toxoid is used as a carrier molecule. The structure of the polysaccharide, in particular its size, therefore has an influence on the immunogenicity of the carrier protein. There is, therefore, no evidence that the immune responses induced by a polysaccharide conjugate can be extrapolated to another conjugate. Schneerson R. et al., (Infection and Immunity (1986); 52, 519-528) shows the presence of an antibody response against the carrier protein in a study of immunogenicity relating to a capsular polysaccharide of serotype 6B pneumococcus conjugated to the tetanus toxoid, however the dose of protein administered (≦80 μg) is comparatively much higher than the usual dose for vaccination against tetanus, between 15 μg and 30 μg, or between 5 and 10 Lf, of tetanus toxoid.

The fact, therefore, of coupling a polysaccharide to a carrier molecule of vaccinal interest, such as the tetanus toxoid or the diphtheria toxoid, leads to an at least partial loss of the immunogenicity of the carrier. A polysaccharide conjugate as such is not sufficient to induce complete immunity with respect to the carrier. The method according to which a polysaccharide or an oligosaccharide is coupled to an antigenic carrier of vaccinal interest does not, therefore, represent a good solution for a person skilled in the art, since it does not make it possible to eliminate the introduction of the free carrier into the vaccine in order to induce complete immunity with respect to this carrier, or requires abnormally high amounts of conjugated carrier.

There exists, therefore, a need to identify a multivalent vaccine composition which is capable of preventing and/or of treating Clostridium tetani and/or Corynebacterium diphtheriae infections at the same time as ailments caused by microorganisms which express polysaccharide structures at their surface, and which satisfies the need to limit the overall antigenic load injected, in order to avoid the negative interactions between the antigens.

### SUMMARY OF THE INVENTION

For this purpose, the present invention relates to the use of a composition comprising n Streptococcus pneumoniae polysaccharides conjugated to the tetanus toxoid and p Streptococcus pneumoniae polysaccharides conjugated to the diphtheria toxoid, for manufacturing a vaccine which protects against Clostridium tetani and/or Corynebacterium diphtheriae infections in which:

(1) n and p are other than 1, with p being, however, <15,

(2) 2≦n+p≦38,

(3) the total amount of conjugated toxoid present in one vaccine dose is sufficient to induce protection against Clostridium tetani and/or Corynebacterium diphtheriae infections.

In one embodiment, the total amount of conjugated tetanus toxoid present in one vaccine dose is <40 μg, preferably between 10 and 25 μg.

In another embodiment, the total amount of conjugated diphtheria toxoid present in one vaccine dose is <130 μg, preferably between 20 and 85 μg.

In another embodiment, n and p are ≦2.

According to this embodiment, the polysaccharides conjugated to the tetanus toxoid are identical to, different from, or partially different from the polysaccharides which are conjugated to the diphtheria toxoid.

In one particular aspect, it relates to the use of a composition according to the invention in which n is equal to 7, the polysaccharides conjugated to the tetanus toxoid consisting of the capsular polysaccharides of the serotypes 1, 4, 5, 7F, 9V, 19F and 23F, and in which p is equal to 4, the polysaccharides conjugated to the diphtheria toxoid consisting of the capsular polysaccharides of the serotypes 3, 6B, 14 and 18C.

In another embodiment, it relates to the use of a composition as described in which p is equal to zero.

In one particular aspect of this embodiment, n is equal to 11 and the polysaccharides are the capsular polysaccharides of the serotypes 1, 3, 4, 5, 6B, 7F, 9V, 14, 18C, 19F and 23F.

In another embodiment, it relates to the use of a composition as described in which n is equal to zero.

In one particular aspect, it relates to the use of a composition according to the invention in which n is equal to zero and p is equal to 11, and in which the polysaccharides are the capsular polysaccharides of the serotypes 1, 3, 4, 5, 6B, 7F, 9V, 14, 18C, 19F and 23F.

The present invention also relates to a method for inducing, in mammals, an immune response to Clostridium tetani and/or to Corynebacterium diphtheriae, which consists in administering a composition as described in its variants above comprising n polysaccharides originating from Streptococcus pneumoniae conjugated to the tetanus toxoid and p polysaccharides originating from Streptococcus pneumoniae conjugated to the diphtheria toxoid.

The present invention also relates to a method for protecting mammals against a Clostridium tetani and/or Corynebacterium diphtheriae infection, in which a composition as described in its variants above comprising n polysaccharides originating from Streptococcus pneumoniae conjugated to the tetanus toxoid and p polysaccharides originating from Streptococcus pneumoniae conjugated to the diphtheria toxoid is administered.

### DETAILED DESCRIPTION OF THE INVENTION

In the context of the present invention, various terms employed are hereinafter defined:

The term “polysaccharide” is intended to mean a polymer comprising a series of several identical or different saccharide molecules which are mutually linked via covalent bonds. This term also encompasses that of “polysaccharide” and of “oligoside”.

The term “toxoid” is intended to mean a toxin which is modified chemically or by genetic engineering (by deletion, substitution or insertion of one or more nucleotides), which has lost its pathogenic power, but which induces an immune response capable of neutralizing the pathogenic power of the natural toxin.

The term “polysaccharide conjugated” or “conjugated polysaccharide” is intended to mean a polysaccharide coupled either to the tetanus toxoid or to the diphtheria toxoid, by means of one or more covalent bonds. According to this definition, any polysaccharide which is conjugated both to the tetanus toxoid and to the diphtheria toxoid is excluded.

The term “dose of vaccine” or “vaccine dose” is intended to mean the amount of the composition according to the invention which is given to humans in one administration.

The phrase “the total amount of conjugated toxoid present in one dose of vaccine is sufficient to induce protection against Clostridium tetani and/or Corynebacterium diphtheriae infections” is intended to mean the total amount of conjugated tetanus toxoid contained in one dose of vaccine, or the total amount of conjugated diphtheria toxoid contained in one half dose of vaccine, which, when it is injected into a guinea pig, in a single administration, in the form of a composition according to the invention, gives it an at least 80% chance of survival at 10 days after a challenge which contains 10 minimum lethal doses (MLDs) of tetanus toxin or 10 MLDs of diphtheria toxin. By way of illustration, if a healthy individual receives 3 doses, at regular intervals, of a vaccine (in the context of a primary vaccination, for example) manufactured using a composition comprising n Streptococcus pneumoniae polysaccharides conjugated to the tetanus toxoid and p Streptococcus pneumoniae polysaccharides conjugated to the diphtheria toxoid in which one vaccine dose contains a total of 10 μg of conjugated tetanus toxoid and 60 μg of conjugated diphtheria toxoid, this amount of conjugated tetanus toxoid will be considered as inducing protection against Clostridium tetani if it is observed that, in a group of guinea pigs immunized with the same composition and each receiving the equivalent of 10 μg of conjugated tetanus toxoid, there is a survival rate at 10 days of at least 80% after injection of 10 MLDs of tetanus toxin into each guinea pig. Similarly, the amount of conjugated diphtheria toxoid contained in said vaccine dose will also be considered as inducing protection against Corynebacterium diphtheriae if it is observed that, in a group of guinea pigs immunized with the same composition and each receiving the equivalent of a total of 30 μg of conjugated diphtheria toxoid, there is a survival rate at 10 days of at least 80% after injection of 10 MLDs of diphtheria toxin into each guinea pig. For the practical details concerning the protection test in guinea pigs, reference may be made to example 2. It is observed that the results of the protection tests carried out in guinea pigs correspond well with the levels of protection obtained in humans.

The term “serotype” or “serogroup” is intended to mean a strain of a bacterial species, defined by the chemical structure of the capsular polysaccharide or by means of the immune serum specific for the capsular polysaccharide.

The invention relates to the use of a combination of at least two Streptococcus pneumoniae polysaccharides coupled to the tetanus toxoid and/or of at least two Streptococcus pneumoniae polysaccharides coupled to the diphtheria toxoid, in a vaccine preparation for inducing immunity which protects against Clostridium tetani and/or Corynebacterium diphteriae, without it being necessary to add thereto, as a supplement, a significant amount of free tetanus and/or diphtheria toxoid.

A composition according to the invention does not need to be combined with tetanus and/or diphtheria toxoid in nonconjugated form and/or in conjugated form originating from another vaccine, at the time of its administration, in order to produce its protective effect with respect to tetanus and/or to diphtheria. A composition according to the invention thus contributes to decreasing the overall vaccine antigenic load administered since it also induces immunity which protects against the various strains of Streptococcus pneumoniae which express at their surface the polysaccharides corresponding to those of the combination of conjugates. This use is the subject of application WO 98/51339.

The prior art teaches that conjugating a carrier molecule, such as the tetanus or diphtheria toxoid, to a polysaccharide or an oligosaccharide causes a loss of immunogenicity of the carrier molecule as a result of the epitopes which induce antibodies which neutralize or induce protective immunity being masked. Consequently, the amount of carrier molecule in the conjugate has to be increased in order to obtain protective immunity which is equivalent to that of the free carrier. Schneerson R. et al., in Infection and Immunity (1986); 52, 519-528), mentions required amounts of tetanus toxoid of between 80 μg and 250 μg in the serotype 6B pneumococcus polysaccharide conjugate, in order to observe an immune response against the tetanus toxoid in humans. Surprisingly, when at least two different pneumococcus polysaccharides are used for the conjugation to a carrier molecule such as the tetanus toxoid, the applicant shows that the total amount of carrier molecule required for inducing protective immunity is clearly lower than that present in a composition containing a single conjugate as mentioned by Schneerson R. et al. Thus, the total amount of conjugated tetanus toxoid included in a combination of at least two different polysaccharide conjugates according to the invention does not need to reach or to exceed 40 μg in order to induce protective immunity against tetanus. Even more surprisingly, this maximum total amount required is lower than the 54 μg total dose of tetanus toxoid included in the DTP-IPV-PRP-T (diphtheria, tetanus, whooping cough, polio, hemophilus conjugate) pentavalent vaccine already commercially available (30 μg of toxoid exists in nonconjugated form and 24 μg exists in a form conjugated to the capsular polysaccharide of hemophilus influenzae). Combining at least two different pneumococcus polysaccharides conjugated to the same carrier protein therefore promotes the development of protective immunity with respect to the carrier.

In order to carry out the conjugation of the tetanus toxoid to the pneumococcus polysaccharides, at least two thereof are chosen from the 23 serotypes 1, 2, 3, 4, 5, 6B, 7F, 8, 9N, 9V, 10A, 11A, 12F, 14, 15B, 17F, 18C, 19F, 19A, 20, 22F, 23F and 33F, but preferably at least two thereof are chosen from those of the group consisting of the 11 serotypes 1, 3, 4, 5, 6B, 7F, 9V, 14, 18C, 19F and 23F. Thus, a composition of polysaccharide conjugates inducing protective immunity with respect to tetanus preferably contains between 2 and 11 different conjugates with a total amount of conjugated tetanus toxoid in one vaccine dose <40 μg, preferably between 10 μg and 25 μg. Preferably, a composition according to the invention consists of 11 capsular polysaccharide conjugates corresponding to the serotypes 1, 3, 4, 5, 6B, 7F, 9V, 14, 18C, 19F and 23F of pneumococcus and contains a total amount of conjugated tetanus toxoid of between 10 μg and 25 μg per vaccine dose. This dose is, very surprisingly, equivalent to that contained, for example, in a conventional vaccine such as DTP (diphtheria, tetanus, polio) in which the tetanus toxoid exists solely in free form at a dose of between 15 and 30 μg or between 5 Lf and 10 Lf (method for quantifying toxoids in flocculation units). The fact that, in a dose of vaccine prepared using a composition with 11 polysaccharide conjugates, there is no need for an amount of conjugated tetanus toxoid which is greater than that present in the DTP vaccine in order to induce protective immunity with respect to tetanus means that the conjugates of the composition mutually cooperate, in a surprising way, so as to cancel out the negative effects of the conjugation on the immunogenicity of the carrier. In addition, the polysaccharide conjugates coupled to the tetanus toxoid according to the invention also contribute to the induction of protective immunity with respect to the corresponding serotypes/serogroups of pneumococcus.

For carrying out the conjugation of a Streptococcus pneumoniae polysaccharide to the diphtheria toxoid, it is necessary to take into account the fact that the amount of diphtheria toxoid required to obtain protection against diphtheria is approximately between 2 and 4 times greater than that required for the tetanus toxoid. The total amount of conjugated diphtheria toxoid per vaccine dose is <130 μg so as not to observe negative interference with the immune response with respect to the pneumococcus polysaccharides, and preferably between 20 and 85 μg. For these reasons, a composition of Streptococcus pneumoniae polysaccharides conjugated to the diphtheria toxoid can contain between 2 and 15 different conjugates without, however, exceeding this value. For the preparation of the diphtheria conjugates, between 2 and 15 different capsular polysaccharides can be chosen from the 23 identified. Preferably, a composition according to the invention consists of 11 capsular polysaccharide conjugates corresponding to the serotypes 1, 3, 4, 5, 6B, 7F, 9V, 14, 18C, 19F and 23F and contains a total amount of conjugated diphtheria toxoid of between 40 μg and 85 μg per vaccine dose. Very surprisingly, the total amount of conjugated diphtheria toxoid in this type of composition is equivalent to that contained, for example, in a conventional vaccine such as DTP (diphtheria, tetanus, polio) in which the diphtheria toxoid exists solely in free form at a dose of between 45 and 90 μg or between 15 Lf and 30 Lf. The conjugates of the composition which are coupled to the diphtheria toxoid mutually cooperate, in an unexplained way, so as to cancel out the negative effects of the conjugation on the immunogenicity of the carrier in particular. In addition, a composition of Streptococcus pneumoniae polysaccharides coupled to the diphtheria toxoid according to the invention also contributes to the induction of protective immunity with respect to the various strains of Streptococcus pneumoniae which express at their surface the polysaccharides corresponding to those of the conjugate composition.

A composition of conjugated polysaccharides according to the invention can also comprise at least two polysaccharides conjugated to the tetanus toxoid and at least two polysaccharides conjugated to the diphtheria toxoid, so as to induce protective immunity with respect to Clostridium tetani and with respect to Corynebacterium diphtheriae. The polysaccharides can all be different and chosen from those corresponding to the 23 serotypes of pneumococcus. In this type of composition, the total number p of diphtheria toxoid conjugates can vary between 2 and 15, for the reasons referred to above, the total number n of tetanus toxoid conjugates being able to vary, itself, between 2- and 23-p, and the total amounts of conjugated tetanus toxoid and diphtheria toxoid contained in one vaccine dose being less than 40 μg and 130 μg, respectively. Preferably, the choice of the polysaccharides is restricted to the serotypes 1, 3, 4, 5, 6B, 7F, 9V, 14, 18C, 19F and 23F of pneumococcus. In this type of composition, since the total number n of polysaccharides conjugated to the tetanus toxoid varies preferably between 2 and 9, and the total number p of diphtheria toxoid conjugates varies, itself, preferably between 2- and 11-n, the total amounts of conjugated tetanus toxoid and diphtheria toxoid are preferably between 10 and 25 μg, and between 20 μg and 85 μg, respectively, per vaccine dose. In a most particularly preferred way, a conjugate composition according to the invention comprises 7 polysaccharides conjugated to the tetanus toxoid, corresponding to the serotypes 1, 4, 5, 7V, 9V, 19F and 23F, and 4 polysaccharides conjugated to the diphtheria toxoid, corresponding to the serotypes 3, 6B, 14 and 18C, the total amounts of conjugated tetanus and diphtheria toxoid contained in a vaccine dose prepared from this composition being between 10 and 15 μg, and between 40 μg and 65 μg, respectively.

One or more polysaccharides in a composition according to the invention can be used for preparing the tetanus toxoid conjugate and the diphtheria toxoid conjugate, provided that the composition respects the conditions of the invention, namely that it contains at least two different tetanus toxoid conjugates and at least two different diphtheria toxoid conjugates. The total number of tetanus toxoid-coupled polysaccharide conjugates and of diphtheria toxoid-coupled polysaccharide conjugates can be 38, 23 conjugates being coupled to the tetanus toxoid and 15 conjugates coupled to the diphtheria toxoid. In a particular aspect, all the polysaccharides used for preparing the tetanus toxoid conjugates are also used for preparing the diphtheria toxoid conjugates. In this case, the vaccine composition contains the same number of tetanus toxoid conjugates and of diphtheria toxoid conjugates, this number being between 2 and 15 since the number of diphtheria toxoid conjugates cannot exceed 15 for reasons of overall antigenic load.

The composition comprising the combination of polysaccharides conjugated to the tetanus toxoid and of polysaccharides conjugated to the diphtheria toxoid induces protective immunity with respect to Clostridium tetani and to Corynebacterium diphtheriae which is the result of that observed with respect to Clostridium tetani, induced by the group of components of the composition consisting of the polysaccharides conjugated to the tetanus toxoid, and of that observed with respect to Corynebacterium diphtheriae, induced by the group of components of the composition consisting of the polysaccharides conjugated to the diphtheria toxoid. In addition, this combination, via the polysaccharides present, also contributes to the induction of protective immunity with respect to the corresponding serotypes of pneumococcus. The fact of mixing polysaccharides conjugated to the tetanus toxoid with polysaccharides conjugated to the diphtheria toxoid does not, insofar as the conditions concerning n and p are respected, cause any appearance of negative interference in the development of the immune response to the various polysaccharides.

The polysaccharides can be advantageously extracted from the various strains of Streptococcus pneumoniae according to conventional methods and purified likewise. These polysaccharides can be used in crude form after extraction/purification; or alternatively they can be fragmented in order to obtain polysaccharides of mean molecular weights lower than those of the polysaccharides of origin. A particularly advantageous fragmenTTion method is described in WO 93/07178, which is incorporated by way of reference.

A conjugate in which a polysaccharide is coupled, by covalent bonding, to a protein can be obtained according to conventional methods well known to a person skilled in the art. Use may be made of linkers or of spacers in order to carry out the conjugation. Depending on the conjugation method used, the conjugate which results therefrom may be a conjugate in which the polysaccharide is linked to the protein via a single chemical function (sun or neoglycoconjugate type) or via several functions (rake and random coil type). It is within the scope of a person skilled in the art to determine the most suitable method of conjugation depending on the nature of the polysaccharide and, more particularly, on the chemical groups carried by the polysaccharide which can be used in the course of the conjugation reaction.

Hereinafter, by way of example, the preparation of various compositions according to the invention is presented, the polysaccharides chosen being derived from the serotypes 1, 3, 4, 5, 6B, 7F, 9V, 14, 18C, 19F and 23F. The polysaccharides derived from these serotypes were fragmented according to the method described in WO 93/07178 and are coupled to the tetanus toxoid (except the polysaccharide of type 1) according to the conjugation method described in WO 93/07178. Briefly, a polysaccharide is subjected to reductive amination in the presence of sodium cyanoborohydride in order to attach a diaminohexane molecule to a reductive end group. Then, the polysaccharide thus derived is activated with a succinimide group using disuccinimidyl suberate (DSS). The polysaccharide thus activated is reacted directly with the carrier protein. The polysaccharide of serotype 1 is coupled to the diphtheria toxoid or to the tetanus toxoid according to the conjugation method described in U.S. Pat. No. 5,204,098, incorporated by way of reference. The experimental conditions were controlled so that conjugates in which the amount of protein represents between 1 and 4 times, preferably twice, the amount of polysaccharide are obtained. Thus, for a polysaccharide conjugate coupled to the tetanus toxoid, 1 μg of a particular polysaccharide can be coupled to approximately 2 μg of tetanus toxoid. At the end of each conjugation reaction, the amount of noncoupled residual tetanus toxoid is very low. The elimination of the residual free toxoid is completed as needed, by dialysis or ultrafiltration. The composition of polysaccharides conjugated to the tetanus toxoid is obtained by mixing the various conjugates with one another such that the amount of polysaccharide contained in each conjugate is at least that necessary to observe protective immunity with respect to the corresponding serotype, and that the total amount of conjugated tetanus toxoid included in a vaccine dose is less than 40 μg, and preferably between 10 and 25 μg. The residual amount of nonconjugated tetanus toxoid in a composition according to the invention is also controlled, by capillary electrophoresis or by chromatography, so that it represents less than 5% of the total amount of conjugated tetanus toxoid. The composition described in example 1.1 is prepared according to the abovementioned operating techniques.

The coupling of the polysaccharides to the diphtheria toxoid was carried out as follows: hydrazide groups are incorporated onto the polysaccharide by reacting the polysaccharide with an excess of adipic acid dihydrazide (ADH) in the presence of ethyldimethylaminopropylcarbodiimide (EDAC) and of sodium cyanoborohydride (for all types except type 3), or simply in the presence of sodium cyanoborohydride (for type 3). The polysaccharide thus derived is left to react with the carrier protein in the presence of EDAC. The experimental conditions were controlled so that conjugates in which the amount of protein represents between 1 and 4 times the amount of polysaccharide are obtained. At the end of each conjugation reaction, the amount of noncoupled residual diphtheria toxoid is very low. The elimination of the residual free toxoid is completed as needed, by dialysis or ultrafiltration. The composition of polysaccharides conjugated to the diphtheria toxoid is obtained by mixing the various conjugates with one another such that the amount of polysaccharide contained in each conjugate is at least that necessary to observe protective immunity with respect to the corresponding serotype, and that the total amount of conjugated diphtheria toxoid included in a vaccine dose is less than 130 μg, and preferably between 20 and 85 μg. The residual amount of nonconjugated diphtheria toxoid in a composition according to the invention is also controlled, by capillary electrophoresis or by high performance liquid chromatography, so that it represents less than 5% of the total amount of conjugated diphtheria toxoid.

The tetanus toxoid and the diphtheria toxoid were prepared by formaldehyde detoxification using toxins extracted from Corynebacterium diphtheriae and from Clostridium tetani, respectively, well known to a person skilled in the art. The diphtheria toxoid can also be a nontoxic mutant of the diphtheria toxin, such as, for example, the compound CRM197. The tetanus and diphtheria toxoids used for preparing the polysaccharide conjugates have a degree of purity of greater than 90%.

A composition according to the invention which comprises both polysaccharide conjugates coupled to the tetanus toxoid and polysaccharide conjugates coupled to the diphtheria toxoid is manufactured by mixing the various polysaccharide conjugates which have been prepared individually, and taking into account the fact that the polysaccharide conjugates coupled to the diphtheria toxoid are proportionally in greater amounts than the polysaccharide conjugates coupled to the tetanus toxoid. Thus, in a composition which comprises as many polysaccharide conjugates coupled to the diphtheria toxoid as conjugates coupled to the tetanus toxoid, the total weight of the conjugates coupled to the diphtheria toxoid is, on average, between 3 and 6 times greater than the total weight of the conjugates coupled to the tetanus toxoid. The preparation of the composition described in example 1.2 is carried out in this way.

A composition according to the invention can be formulated with a diluent or support which is acceptable from a pharmaceutical point of view, e.g. an aluminum hydroxide, an aluminum phosphate or an aluminum hydroxyphosphate, and, where appropriate, a lyophilization excipient. In general, these products can be selected as a function of the method and route of administration and according to standard pharmaceutical practices. The suitable diluents, as well as that which is essential for the development of a pharmaceutical composition, are described in Remington's Pharmaceutical Sciences, a standard reference book in this field.

A composition according to the invention advantageously contains a phosphate buffer and sodium chloride, and can be adjuvanted using aluminum hydroxide. A preservative, such as phenoxyethanol formol can also be used. A vaccine dose can be prepared in a volume of 0.1 ml to 2 ml, and preferably in a volume of 0.5 ml, and can contain 0.475 mg of PO₄⁻² ion, 4.5 mg of sodium chloride and optionally 300 μg of AL³⁺ ions.

The invention also relates to a method for protecting against a Clostridium tetani and Corynebacterium diphtheriae infection in humans, in which a composition comprising at least two different polysaccharides originating from Streptococcus pneumoniae, conjugated to the tetanus toxoid, and at least two different Streptococcus pneumoniae polysaccharides, conjugated to the diphtheria toxoid, is administered. If the desire is to limit the prevention method to just one of the 2 infections, a composition comprising at least two polysaccharides originating from Streptococcus pneumoniae, conjugated to one of the two toxoids, either to the tetanus toxoid or to the diphtheria toxoid, is then used. The method for preventing the Clostridium tetani and Corynebacterium diphtheriae infections can be applied both to adult or elderly human beings and to young children or infants.

The protection method according to the invention is implemented by administering at least one vaccine dose of the composition according to the invention. For example, between 1 and 3 injections can be given, but preferably 3 injections are given while respecting a one month time delay between each injection. A composition according to the invention can be administered via any conventional route used in the field of vaccines, in particular via the systemic route, i.e. the parenteral route, e.g. via the subcutaneous, intramuscular, intradermal or intravenous route; or via the mucosal route, e.g. via the oral or nasal route. The amount administered takes into account various parameters, in particular the number of conjugates present in the composition, the nature of the polysaccharides used, the type of carrier(s) used or the route of administration. The dose of polysaccharide required, contained in each conjugate, in order to observe protective immunity with respect to the corresponding serotype consecutive to parenteral administration is generally between 0.5 μg and 10 μg; but preferably between 0.5 μg and 5 μg, and even more preferably between 0.5 μg and 2 μg for conjugates which are coupled to the tetanus toxoid.

### EXAMPLE 1

### Composition of Various Compositions of Streptococcus pneumoniae Capsular Polysaccharides Coupled to the Tetanus Toxoid (TT) and/or to the Diphtheria Toxoid (DT)

1.1: Composition of a human vaccine dose of a tetravalent composition consisting of the capsular polysaccharides of the serotypes 23F, 14, 19, 6B conjugated to the tetanus toxoid

| | Amount of | Amount of |
|---|---|---|
| | polysaccharide for | conjugated TT for |
| | one human vaccine | one human vaccine |
| Serotype | dose (μg) | dose (μg) |
| 23F | 1 | 1.5 |
| 14 | 1 | 2 |
| 19F | 1 | 1.5 |
| 6B | 1 | 1.5 |

The total amount of conjugated polysaccharide in the composition is 4 μg.

The total amount of conjugated TT in the composition is 6.5 μg.

TT=tetanus toxoid.

The total volume of a vaccine dose is 0.5 ml.

1.2: Composition of a human vaccine dose of an 11-valence composition consisting of the capsular polysaccharides of the serotypes 1, 4, 5, 7F, 9V, 19F and 23F conjugated to the tetanus toxoid and of the 5 capsular polysaccharides of the serotypes 3, 6B, 14, 18C conjugated to the diphtheria toxoid

| | Amount of | Amount of TT | Amount of DT |
|---|---|---|---|
| | polysaccharide | for one human | for one human |
| | for one human | vaccine dose | vaccine dose |
| Serotype | vaccine dose (in μg) | (in μg) | (in μg) |
| 1 | 1 | 2.7 | |
| 3 | 3 | | 15 |
| 4 | 1 | 1.7 | |
| 5 | 1 | 1 | |
| 6B | 10 | | 31 |
| 7F | 1 | 1.3 | |
| 9V | 1 | 1.6 | |
| 14 | 3 | | 8 |
| 18C | 3 | | 6 |
| 19F | 1 | 2.5 | |
| 23F | 1 | 1.2 | |

The total amount of conjugated polysaccharide in a vaccine dose of this composition is 26 μg.

The total amount of conjugated DT in a vaccine dose of this composition is 60 μg.

The total amount of conjugated TT in a vaccine dose of this composition is 12 μg.

DT=diphtheria toxoid.

The total volume of a vaccine dose is 0.5 ml.

### EXAMPLE 2

### Protection of Guinea Pigs Against Tetanus and/or Diphtheria After Injection of a Composition of Streptococcus pneumoniae Capsular Polysaccharides Coupled to the Tetanus Toxoid (TT) and/or to the Diphtheria Toxoid (DT)

The compositions of Streptococcus pneumoniae capsular polysaccharides coupled to the tetanus toxoid (TT) and/or to the diphtheria toxoid (DT), described in example 1, were tested in guinea pigs for their capacity to protect these animals against tetanus or diphtheria. Bivalent compositions of polysaccharides coupled to the tetanus toxoid, including that consisting of the capsular polysaccharides 4 and 19F, but also bivalent compositions of polysaccharides coupled to the diphtheria toxoid, in particular the composition consisting of the capsular serotypes 6B and 14, were also tested. 11-valent compositions of polysaccharides coupled to the tetanus toxoid, including that consisting of the capsular polysaccharides 1, 3, 4, 5, 6B, 7F, 9V, 14, 18C, 19F and 23F were also tested. The corresponding 11-valent compositions of polysaccharides coupled to the diphtheria toxoid were also studied. A 15-valent composition consisting of the capsular polysaccharides of the serotypes 1, 4, 5, 6B, 7F, 9V, 18C, 19F and 23F, conjugated to the tetanus toxoid and of the capsular polysaccharides of the serotypes 3, 6B, 9V, 14, 18C, 23F, conjugated to the diphtheria toxoid, was tested. Finally, a 15-valent composition consisting of the capsular polysaccharides of the serotypes 1, 3, 4, 5, 6B, 7F, 8, 9V, 12F, 14, 15, 18C, 19F, 22F and 23F, which are all coupled to the tetanus toxoid, and the same composition of polysaccharides which are coupled to the diphtheria toxoid, was tested. All these compositions are in liquid medium. The excipient used contains a phosphate buffer and sodium chloride.

Groups of 10 guinea pigs are formed for each composition tested. The animals receive, via the subcutaneous route, ⅓ of a total human vaccine dose when it is planned then to challenge them with the tetanus toxin. The total human vaccine dose is, in fact, the sum of the 3 vaccine doses that the individual receives in a scheme of primary vaccination with 3 injections. Each guinea pig receives in fact an injection of a human vaccine dose in 0.5 ml. The animals receive, via the subcutaneous route, ⅙ of a total human vaccine dose when it is planned then to challenge them with the diphtheria toxin, which corresponds to an injection of half a human vaccine dose in 0.25 ml. This corresponds, for example, to the administration of one vaccine dose of the 11-valence composition as described in example 1.2, for the animals challenged with the tetanus toxin, and to the administration of half a vaccine dose of the 11-valence composition as described in example 1.2, for the animals challenged with the diphtheria toxin.

30 days after the injection of the vaccine compositions tested, the immunized animals are challenged by subcutaneous injection into each animal of 10 minimum lethal doses (MLDs) of tetanus toxin or of 10 MLDs of diphtheria toxin. In parallel, two control groups of nonimmunized animals consisting of 2 and 3 guinea pigs are challenged with 1 MLD of tetanus toxin and 1 MLD of diphtheria toxin, respectively. These control groups are used to validate the MLD of the 2 toxins on the control animals, which must all be dead within the 96 hours which follow the challenge. The dead animals in the other groups of challenged immunized guinea pigs are also counted. The vaccine combination tested is considered to be protective with respect to tetanus and/or to diphtheria if an 80% minimum survival rate is noted at the end of 10 days. The survival studies carried out in the guinea pigs immunized with the various compositions of polysaccharide conjugates mentioned show a survival rate greater than 80% after challenge with the tetanus or diphtheria toxin.

### EXAMPLE 3

### Study of the Protective Activity of a Pool of Immune Sera Obtained from Guinea Pigs Immunized with an 11-Valent Composition Adjuvanted with Alum

The composition as described in example 1.2 mixed with an alum gel was tested using a slightly different protection test. The preparation of the mixture is described in example 4.

Depending on whether it is desired to evaluate the protective power of this adjuvanted formulation with respect to a Clostridium tetani or Corynebacterium diphtheriae infection, the animals challenged with a mixture consisting of guinea pig immune sera and of toxin, either tetanus or diphtheria toxin, are mice or guinea pigs, respectively, the first part of the experimental protocol remaining the same in the two cases. The test as carried out in mice takes into account the recommendations published by the NIH in a 4th revision carried out on Dec. 15, 1952. For the test carried out in guinea pigs, account is taken of the recommendations of the NIH published in a 4th revision dated Mar. 01/1947.

In a first part, specifically, a group of guinea pigs is immunized with half a total human vaccine dose of the adjuvanted composition. The immune sera of each guinea pig are collected and grouped together in a single pool.

In order to evaluate the protective activity of this pool of serum in mice, the pool is diluted 10-fold and a reference calibration serum having a titer of 0.1 IU/ml of anti-tetanus antibodies is prepared. The pool of serum is then titrated for anti-tetanus antibodies by seroneutralization in vivo in mice using the reference calibration serum. It is verified beforehand that the injection, in a volume of 0.5 ml, of a mixture consisting of 0.01 IU of the calibration serum and of a lethal dose 100 of tetanus toxin, in the tail vein of each mouse, provokes the death, within 96 hours, of all the mice which have received this mixture. The pool of serum is considered to be protective and, as a consequence, the formulation is considered to be adjuvanted, if it has a titer >2 IU/ml.

In order to evaluate the protective capacity of the pool of serum in guinea pigs, the pool of serum is titrated for anti-diphtheria antibodies by seroneutralization in vivo in guinea pigs using a reference anti-diphtheria serum with a titer of 6 IU/ml. It is verified beforehand that the subcutaneous injection, in a volume of 3 ml, of a mixture which contains 1 U of the reference anti-diphtheria serum and one lethal dose 100 of diphtheria toxin kills, within 96 hours, all the guinea pigs which have received this mixture. The titer of the pool of serum, which must be greater than 2 IU/ml in order to be protective, is then evaluated.

The pool of sera obtained using the adjuvanted formulation tested has an anti-tetanus and anti-diphtheria antibody titer greater than 2 IU/ml, evaluated in the seroneutralization tests in vivo in mice or guinea pigs.

### EXAMPLE 4

### Study of the Immune Response Against Tetanus Toxoid and/or Against Diphtheria Toxoid After Injection of a Composition of Streptococcus pneumoniae Capsular Polysaccharides Coupled to the Tetanus Toxoid (TT) and/or to the Diphtheria Toxoid (DT)

Various compositions of Streptococcus pneumoniae capsular polysaccharides coupled to the tetanus toxoid (TT) and/or to the diphtheria toxoid (DT) were tested for their capacity to induce a specific antibody response directed against the tetanus toxoid and/or the diphtheria toxoid. Included in these studies were in particular the combinations mentioned in example 2.

In Monkeys

2 groups of two macaque monkeys (Macaca fascicularis) receive, in a volume of 0.65 ml, via the intramuscular route, 4 weeks apart, 2 injections of an 11-valent combination described in example 1.2 possibly adjuvanted with an alum gel. The dose administered at each injection corresponds to a human vaccine dose (the diluent used is a 10 mM phosphate buffer, pH 6.8, prepared in 0.9% sodium chloride). The adjuvanted combination is prepared by mixing a human vaccine dose with a volume of alum gel containing the equivalent of 300 μg of Al³⁺. Blood samples were taken on the day of the first immunization (D0), the day of the second immunization (D28) and 4 weeks after the second immunization (D56), in order to analyze the content of specific antibodies directed against the tetanus toxoid and the diphtheria toxoid. Specific IgG antibody titers are evaluated by ELISA. In a conventional and known way, the assay is carried out by coating microplates with the aid of a solution of diluted purified tetanus or diphtheria toxoid, followed by a plate saturation phase. For each monkey serum tested, a dilution range is then prepared, which is deposited into the microwells. In parallel, a reference range is prepared from a pool of human immunosera, and the quality controls (comprising in particular anti-tetanus and anti-diphtheria sera with known titers, originating from the National Institute for Biological Standard and Control) are prepared. After another incubation phase, followed by several washes to remove nonspecific antibodies, a volume of a diluted solution of an anti-human-IgG monoclonal antibody coupled to peroxidase, which also cross reacts with macaque IgGs, is deposited into each well. After incubation of the conjugate, followed by washes and revelation of the attached conjugate by coloration using O-phenylenediamine, the intensity of the coloration of each well is measured by spectrophotometric reading.

The results are expressed in international units per ml (IU/ml).

The table below gives the values of the levels of specific antibodies obtained with the 11-valence combination described in example 1.2, possibly adjuvanted with alum gel.

| | | Anti-diphtheria IgGs | | | Anti-tetanus IgGs | | |
|---|---|---|---|---|---|---|---|
| | | IU/ml | | | IU/ml | | |
| | | D0 | D30 | D60 | D0 | D30 | D60 |
| Without | MON- | 0.181 | 6.375 | 5.461 | 0.916 | 49.135 | 35.843 |
| adju- | KEY 1 | | | | | | |
| vant | | | | | | | |
| | MON- | 0.033 | 0.702 | 0.398 | 0.048 | 0.699 | 0.773 |
| | KEY 2 | | | | | | |
| + alum | MON- | 0.360 | 8.22 | 7.708 | 0.921 | 32.506 | 28.324 |
| | KEY 3 | | | | | | |
| | MON- | 0.414 | 16.469 | 10.173 | 0.891 | 19.667 | 20.851 |
| | KEY 4 | | | | | | |

The results show a very clear increase in the level of specific anti-tetanus and anti-diphtheria antibodies. An adjuvant effect of the alum gel is also noted since the specific antibody titers are higher.

In Humans

A group of 12 healthy adults received 2 vaccine doses, 4 weeks apart, of a tetravalent vaccine combination as described in example 1.1. Blood samples were taken before the first immunization (D0), on the day of the second immunization (D28) and 4 weeks after the second immunization (D56), in order to analyze the content of specific antibodies directed against the tetanus toxoid. The specific antibodies of type IgG are assayed by ELISA as described above.

On D0, the mean titer of antibodies specific for the tetanus toxoid is 5.01 IU/ml. This titer increases to 9.15 IU/ml on D28. The second immunization has no effect on the anti-tetanus antibody titer (on D56, it is 8.71 IU/ml). The vaccine combination induces, therefore, a specific immune response with respect to the carrier molecule. Similar immunogenicity studies have been carried out, in infants, young children and elderly individuals, with other compositions, in particular those mentioned in example 2. They also show that these combinations induce specific immune responses directed against the tetanus toxoid and/or the diphtheria toxoid.

1. The use of a composition comprising n Streptococcus pneumoniae polysaccharides conjugated to the tetanus toxoid and p Streptococcus pneumoniae polysaccharides conjugated to the diphtheria toxoid, for manufacturing a vaccine which protects against Clostridium tetani and/or Corynebacterium diphtheriae infections in which:
(1) n and p are other than 1, with p being, however, <15,
(2) 2≦n+p≦38,
(3) the total amount of conjugated toxoid present in one vaccine dose is sufficient to induce protection against Clostridium tetani and/or Corynebacterium diphtheriae infections.

2. The use according to claim 1, in which the total amount of conjugated tetanus toxoid present in one vaccine dose is <40 μg, preferably between 10 and 25 μg.

3. The use according to claim 1 or 2, in which the total amount of conjugated diphtheria toxoid present in one vaccine dose is <130 μg, preferably between 20 and 85 μg.

4. The use according to any one of claims 1 to 3, in which n and p are ≦2.

5. The use according to claim 4, in which the polysaccharides conjugated to the tetanus toxoid are all different from, partially different from, or identical to the polysaccharides which are conjugated to the diphtheria toxoid.

6. The use according to claim 5, in which n is equal to 7, the polysaccharides which are conjugated to the tetanus toxoid consisting of the capsular polysaccharides of the serotypes 1, 4, 5, 7F, 9V, 19F and 23F, and in which p is equal to 4, the polysaccharides which are conjugated to the diphtheria toxoid consisting of the capsular polysaccharides of the serotypes 3, 6B, 14 and 18C.

7. The use according to either of claims 1 and 2, in which p is equal to zero.

8. The use according to claim 7, in which n is equal to 11 and the polysaccharides are the capsular polysaccharides of the serotypes 1, 3, 6B, 4, 5, 7F, 9V, 14, 18C, 19F and 23F.

9. The use according to either of claims 1 and 3, in which n is equal to zero.

10. The use according to claim 9, in which p is equal to 11 and the polysaccharides are the capsular polysaccharides of the serotypes 1, 3, 6B, 4, 5, 7F, 9V, 14, 18C, 19F and 23F.
